(19) United States
(12) Patent Application Publication
(10) Pub. No.: US 2022/0362239 A1
(43) Pub. Date: Nov. 17, 2022

(54) PHARMACEUTICAL COMPOSITION AND METHOD FOR TREATMENT OF ACUTE RESPIRATORY DISTRESS SYNDROME (ARDS) IN CORONAVIRUS DISEASE (COVID-19)

(71) Applicant: PHARMAZZ, INC., Willowbrook, IL (US)

(72) Inventor: Anil Gulati, Naperville, IL (US)

(73) Assignee: PHARMAZZ, INC., Willowbrook, IL (US)

(21) Appl. No.: 17/741,719

(22) Filed: May 11, 2022

Related U.S. Application Data
(60) Provisional application No. 63/187,077, filed on May 11, 2021.

(51) Int. Cl. A61K 31/496; A61K 31/58

(52) U.S. Cl. CPC A61K 31/496; A61K 31/58

(57) ABSTRACT

A pharmaceutical composition for treating acute respiratory distress syndrome (ARDS), multiple end organ failure and survival in serious and life-threatening condition in patients with coronavirus disease 2019 (COVID-19) including (a) centhaquine; (b) antiviral therapies for SARS-CoV-2 infection (remdesivir, ivermectin, chloroquine, hydroxychloroquine, azythromicyn, tenofovir, emtricitabine, ritonavir, lopinavir, ASC09, favipiravir, danoprevir, angiotensin-converting enzyme inhibitors (ACEI), angiotensin receptor blockers (ARB), recombinant human angotensin-converting enzyme 2 (rhACE2), xiyanping, alpha-interferon, fludase (DAS181), eicosapentaenoic acid free fatty acid (EPA-FFA), nitric oxide, PUL-042, Pam2CSK4 acetate, agonists of TLR2 TLR6, and TLR9), convalescent plasma, stem cells or their exosomes, immunomodulation and cytokine-targeted therapies (itolizumab, tocilizumab, sarilumab, acalabrutinib, piclidenoson, tradipitant, CD24Fc, emapalumab, anakinra, TJ003234, BLD-2660, blood purification systems, Spectra Optia Apheresis System, corticosteroids) oxygen concentrator and generator, T89, dantonic, plasminogen supplementation, plasminogen activators, alteplase; (c) budesonide, supportive therapies to reduce fever (like acetaminophen or ibuprofen), steroids (dexamethasone, prednisolone), anticoagulants (aspirin, heparin, non-heparin anticoagulants such as argatroban, bivalirudin, danaparoid, fondaparinux or a direct oral anti-coagulant (DOAC), (d) inhaled synthetic surfactant, antibody to endotoxin, interferon-beta-1a, IV prostaglandin E1, neutrophil elastase inhibitors, nitric oxide and (e) an excipient. A method for preparing the composition for treating acute respiratory distress syndrome, multiple end organ failure and shock symptoms caused by coronaviruses infection, in particular SARS-CoV-2, MERS-CoV and SARS-CoV, using centhaquine and its analogues compound by mechanism of reduction of edema in the lungs, improvement in ratio of arterial partial pressure of oxygen to fraction of inspired oxygen (PaO2/FiO2 or SpO2/FiO2), blood oxygen saturation (SpO2), normalization in respiratory rate, reduction in lung infiltration, improvement in ARDS score, MODS, Ordinal Scale of COVID-19, and better blood flow and oxygenation of tissues.

### CROSS-REFERENCE TO RELATED APPLICATIONS

This application claims priority to and the benefit of U.S. Provisional Application No. 63/187,077 filed on May 11, 2021, the disclosure of which is incorporated herein by reference in its entirety.

### FIELD

The aspects of the disclosed embodiments relates to methods and compositions for reduction of edema in the lungs, improvement in ratio of arterial partial pressure of oxygen to fraction of inspired oxygen (PaO2/FiO2 or SpO2/FiO2), blood oxygen saturation (SpO2), improvement in Ordinal Scale of COVID-19, normalization in respiratory rate, reduction in lung infiltration, improvement in ARDS score, MODS and better blood flow and oxygenation of tissues, thereby treating or preventing acute respiratory distress syndrome (ARDS), multiple end organ failure and shock symptoms in coronavirus disease (COVID-19) and other diseases causing ARDS. Thus far, the only treatment found to improve survival in ARDS is a mechanical ventilation strategy using low tidal volumes (6 mL/kg based upon ideal body weight). In particular, the present disclosure discloses a method and pharmaceutical composition comprising centhaquine in a predefined amount and its analogues and/or administering antiviral therapies, convalescent plasma, stem cells or their exosomes, immunomodulation and cytokine-targeted therapies, blood purification systems, oxygen concentrator and generator, plasminogen supplementation, plasminogen activators, anticoagulants, steroids, inhaled synthetic surfactant, antibody to endotoxin, interferon-beta-1a, IV prostaglandin E1, neutrophil elastase inhibitors, nitric oxide for treating ARDS, multiple end organ failure and shock symptoms caused by coronaviruses infection, in particular SARS-CoV-2, MERS-CoV and SARS-CoV.

### BACKGROUND

Human coronavirus (CoV) infections have traditionally caused a low percentage of annual respiratory infections. There are HCoV-0C43, HCoV-229E, HCoV-NL63 and HCoV-HKU1, which cause mild respiratory illness (1, 2). Over the past two decades, two novel coronaviruses, severe acute respiratory syndrome CoV (SARS-CoV) and Middle East respiratory syndrome CoV (MERS-CoV), have emerged and caused severe human diseases (3, 4). The 2019-nCoV infection is of clustering onset and is more likely to affect older males with comorbidities and can result in severe and even fatal respiratory diseases such as acute respiratory distress syndrome (ARDS).

Severe acute respiratory syndrome coronavirus 2 (SARS-CoV-2) infection leading to coronavirus disease 2019 (COVID-19) has become a global pandemic. COVID-19 illness can manifest from mild disease to severe life-threatening stage involving severe pneumonia and acute respiratory distress syndrome (ARDS) requiring admission in the intensive care unit (ICU). In a review by the WHO-China Joint Mission of 55,924 laboratory-confirmed cases in China 6.1% were classified as critical and 13.8% as severe. Critical stage is when there is respiratory failure, shock, and multiple organ dysfunction or failure. Severe disease is defined as dyspnea, respiratory frequency ≥30 breaths per minute, blood oxygen saturation (SpO2)≤93%, ratio of arterial partial pressure of oxygen to fraction of inspired oxygen (PaO2/FiO2)<300, or lung infiltrates >50% within 24 to 48 hours. Thus, a total of about 20% of patients were in life threatening situation. About 25% of severe and critical cases required mechanical ventilation while the remaining 75% needed only oxygen supplementation.

An increasing incidence of COVID-19 illness is challenging healthcare providers to come up with appropriate treatment decisions. At present there is no widely accepted standard of care regarding pharmacotherapy of patients with COVID-19. It is an unmet medical need and it is critical that potential treatment strategies are identified on a priority basis. There are numerous novel agents that are either in clinical trials or are available through emergency or compassionate use. Most of these agents are repurposed antiviral agents and immune modulating therapies (5). On Mar. 31, 2020 the U.S. Food and Drug Administration in a news release mentioned that there many therapeutic areas being evaluated, including antiviral drugs like remdesivir that might treat the specific virus, as well as host targets, such as interleukin-6 (IL-6) receptor inhibitors that may be helpful in reducing lung inflammation and improving lung function in COVID-19 patients. FDA is also interested in examining whether therapies such as convalescent plasma and hyperimmune globulin, antibody-rich blood products that are taken from blood donated by people who have recovered from the virus, could shorten the length or lessen the severity of the illness. FDA is also working to evaluate whether existing therapies such as chloroquine and hydroxychloroquine help treat patients with COVID-19. In addition, pharmaceutical and biotech companies in China have been gearing up to repurpose existing drugs as treatments for the coronavirus outbreak.

Coronavirus disease (COVID-19), which appeared in December 2019, presents a global challenge, particularly in the rapid increase of critically ill patients with pneumonia and absence of definitive treatment. As of Mar. 19, 2020, over 241,000 cases have been confirmed, with over 9980 deaths. The mortality appears to be around 3-4%; early published data indicate 25.9% with SARS-CoV-2 pneumonia required ICU admission and 20.1% developed ARDS (6).

Vaccines

Several companies are working on the idea of using formulations of RNA or DNA that when injected into the body will initiate cells making a protein used by SARS-CoV-2 (7). A DNA vaccine candidate, INO-4800, designed to prevent COVID-19 infection is being developed by INOVIO Pharmaceuticals, Inc. An open-label trial to evaluate the safety, tolerability and immunological profile of INO-4800 administered by intradermal injection followed by electroporation in healthy adult volunteers is in progress (NCT04336410). Moderna Therapeutics and CureVac are moving fast with DNA and RNA vaccines against COVID-19 in human testing. Moderna's mRNA-1273 is a novel lipid nanoparticle (LNP)-encapsulated mRNA-based vaccine that encodes for a full-length, prefusion stabilized spike (S) protein of SARS-CoV-2. A phase I study of mRNA-1273 sponsored by National Institute of Allergy and Infectious Diseases (NIAID) has begun at Emory University in Atlanta. (NCT04283461). BioNTech (partnered with Pfizer) and CureVac are set to start humans testing, of vaccines developed using messenger RNA, within the coming weeks. BioNTech will manufacture its vaccine, BNT162, at its European mRNA manufacturing facilities with the support of its CDMO partner Polymun. However, no mRNA vaccine is in the market, making this approach more of an unknown. Vaccines using the synthetic biology approach contain synthetic strands of RNA or DNA that code for protein molecules on the surface of the virus. The Bill and Melinda Gates Foundation and the National Institute of Health (NIH) are betting on synthetic biology to engineer new vaccines against the COVID-19 virus. A single-center, open-label, dose-escalating phase I clinical trial in healthy subjects is being conducted to assess the safety, reactogenicity and immunogenicity of recombinant novel coronavirus vaccine (Adenovirus Type 5 Vector (Ad5-nCoV)) manufactured by Beijing Institute of Biotechnology and CanSino Biologics Inc. (NCT04313127). A randomized, double-blinded and placebo-controlled trial in healthy adults (500 subjects) to evaluate the immunogenicity and safety of Ad5-nCoV which encodes for a full-length spike (S) protein of SARS-CoV-2 is ongoing (NCT04341389). Symvivo Corporation is evaluating the safety, tolerability and immunogenicity of bacTRL-Spike vaccine for prevention of COVID-19 (NCT04334980). Johnson & Johnson revealed a lead COVID-19 vaccine candidate that is being developed in partnership with the U.S. Biomedical Advanced Research and Development Authority.

The race to find and produce a safe and effective vaccine is on and optimistically one could be available in 12-18 months. It is possible that multiple vaccines against COVID-19 could be available on a limited basis by second quarter of next year. However, the warnings are that it may not be possible to produce enough vaccine to accommodate the demand (7).

Antiviral Agents

Antiviral therapies for SARS-CoV-2 infection are under intense investigation. Remdesivir and chloroquine have been shown to effectively inhibit SARS-CoV-2 in vitro (8). Remdesivir, a nucleotide analogue prodrug that inhibits viral RNA polymerases, has shown in vitro activity against SARS-CoV-2 (9, 10). A phase III randomized study in 2400 patients to evaluate the safety and antiviral activity of remdesivir with severe COVID-19 is ongoing (NCT04292899). Gilead Sciences is also conducting a trial in 1600 patients with the primary objective to evaluate the efficacy of two regimens of remdesivir compared to standard of care, where clinical status assessment will be done on 11th day of treatment in moderate COVID-19 patients (NCT04292730). In addition, the U.S. National Institute of Allergy and Infectious Diseases has initiated a phase II adaptive, randomized, double-blind, placebo-controlled trial to evaluate remdesivir as a potential treatment for hospitalized adult patients diagnosed with COVID-19 (NCT04280705). Gilead provided remdesivir on a compassionate-use basis to patients hospitalized with confirmed COVID-19 and clinical improvement was observed in 36 of 53 patients (68%) and 7 of the 53 patients (13%) died (11).

Chloroquine is approved as an antimalarial and autoimmune disease drug, however, in vitro testing showed that chloroquine acts as an endosomal acidification fusion inhibitor and blocked infection of a clinical isolate of SARS-CoV-2. Results showing promising in vitro activity of chloroquine against SARS-CoV-2 (8), promoted pilot clinical study to determine efficacy of this drug in COVID-19 patients with different levels of severity. A study conducted in France where confirmed COVID-19 patients were included in a single arm protocol to receive 600 mg of hydroxychloroquine daily and their viral load in nasopharyngeal swabs was tested daily in a hospital setting. Twenty cases were treated in this study and showed a significant reduction of the viral carriage at day 6 post inclusion compared to controls (12). Although these results appear positive, but the study excluded six patients in the hydroxychloroquine arm because they did not complete the study: one patient died, three were transferred to the ICU, and two withdrew. On the other hand, none of the 16 patients in the control group died, withdrew, or needed care in an ICU. An exploratory study is to be conducted in 400 patients to evaluate the efficacy of hydroxychloroquine and azithromycin to treat moderate to severe COVID-19 pneumonia (NCT04329572). A randomized double-blind placebo-controlled clinical trial to determine hydroxychloroquine for chemoprophylaxis in healthcare workers exposed to COVID-19 is being conducted (NCT04328285). A double blind randomized clinical trial has been designed to evaluate the efficacy of hydroxychloroquine as treatment for COVID-19. The investigators hypothesize that a 400 mg per day dose of hydroxychloroquine for 10 days will reduce all-cause hospital mortality in patients with severe respiratory COVID-19 disease (NCT04315896). A triple blinded, phase III randomized controlled trial with parallel groups (200 mg of hydroxychloroquine per day vs. placebo) is aiming to prove safety and efficacy of hydroxychloroquine as prophylaxis treatment for healthcare personnel exposed to COVID-19 patients (NCT04318015). Sanofi has sponsored a clinical study to assess the effect of hydroxychloroquine versus placebo on nasopharyngeal SARS-CoV-2 viral load in outpatient adults with COVID-19 (NCT04333654). A study to evaluate the effectiveness and safety of hydroxychloroquine combined with azithromycin compared to hydroxychloroquine monotherapy in patients hospitalized with pneumonia by SARS-CoV2 virus (NCT04321278) is ongoing and another study to determine efficacy of hydroxychloroquine and azytromicyn for COVID-19 infection in hospitalized but noncritical patients (NCT04322123) is in progress.

The US Food and Drug Administration has authorized clinicians to prescribe chloroquine and hydroxychloroquine for patients admitted to hospital with covid-19. In the emergency use authorization issued on Mar. 28, 2020 the agency acknowledged that the approval was based on “limited in-vitro and anecdotal clinical data.” Under this emergency use authorization chloroquine and hydroxychloroquine can only be used in a hospital setting to treat COVID-19 in adults weighing at least 50 kg. France is another country that permits use of chloroquine and hydroxychloroquine for COVID-19 patients. However, there is opposition from European Medicines Agency and WHO.

A study which aims to assess the efficacy of a daily single dose of tenofovir disoproxil fumarate (TDF) (245 mg)/Emtricitabine (FTC) (200 mg), a daily single dose of hydroxychloroquine (HC) (200 mg), a daily single dose of TDF (245 mg)/FTC (200 mg) plus HC (200 mg) versus placebo, during 12 weeks in (1) reducing the incidence of symptomatic disease and (2) reducing clinical severity COVID-19 among hospital healthcare workers aged 18 to 65 years in public and private hospitals will be conducted in Spain (NCT04334928). AbbVie has sponsored a study where lopinavir/ritonavir will be administered 400 mg/100 mg orally (or weight-based dose adjustment for children) for a 14-day course, or until discharge from hospital, whichever occurs first. This study will have 2-arms in a 1:1 ratio randomization to either the control arm, consisting of standard of care supportive treatment for COVID-19, or the investigational product, lopinavir/ritonavir plus standard of care (NCT04330690). Ascletis Pharma has applied to the Chinese authorities to test two HIV protease inhibitors (ritonavir and ASC09) to treat COVID-19. They are conducting a randomized, open-label, multi-center trial to evaluate the safety and efficiency of ASC09/ritonavir and lopinavir/ritonavir in pneumonia caused by COVID-19 (NCT04261907). Favipiravir, also known as T-705 or Avigan, is a pyrazine derivative that acts as an inhibitor of viral RNA-dependent RNA polymerase (13). It has demonstrated activity against influenza viruses and has been approved in Japan and China for the treatment of novel influenza virus infections and is therefore an attractive candidate for study in patients with COVID-19. Danoprevir, an oral Hepatitis C virus protease inhibitor, approved in China in June 2018 is being investigated to evaluate its efficacy and safety in hospitalized patients infected with SARS-CoV-2 (NCT04291729).

SARS-CoV-2 uses the receptor angiotensin-converting enzyme (ACE) 2 for entry into target cells (Hoffmann et al., 2020) and that both ACEI and ARB could significantly increase mRNA expression of cardiac ACE2 (14). The use of ACEIs/ARBs in patients with COVID-19 or at risk of COVID-19 infection is currently a subject of intense debate. A multicenter, double-blind, placebo-controlled phase II randomized clinical trial of starting losartan in patients with COVID-19 in outpatient settings (NCT04311177) and in inpatient settings (NCT04312009) is currently being planned. In addition, Apeiron Biologics is starting a study using recombinant human angotensin-converting enzyme 2 (rhACE2) as a treatment for patients with COVID-19 to block viral entry and decrease viral replication (NCT04335136).

Jiangxi Qingfeng Pharmaceutical Co. Ltd. Xiyanping injection has anti-inflammatory and immune regulation effects. A randomized, parallel controlled clinical study to treat patients with COVID-19 infection is in progress at multiple centers to determine the efficacy and safety of Xiyanping (NCT04295551). The study design has two groups having lopinavir/ritonavir tablets with (experimental) or without (control) Xiyanping. Another clinical trial is also planned to determine safety and efficacy of Xiyanping in patients with coronavirus infection pneumonia. In this study lopinavir/ritonavir, alpha-interferon nebulization is the comparator group, while experimental group will receive lopinavir/ritonavir, alpha-interferon inhalation plus Xiyanping injection (NCT04275388). Ansun BioPharma of San Diego, Calif. is developing lead candidate Fludase (DAS181), which has shown potential for the treatment of parainfluenza, influenza and other viruses is being tried in severe COVID-19 patients on compassionate use basis (NCT04324489). Eicosapentaenoic acid free fatty acid (EPA-FFA), an omega-3 fatty acid, is being developed by S.L.A. Pharma AG and planning to conduct a randomized controlled study to treat hospitalized subjects with confirmed SARS-CoV-2 (NCT04335032).

Nitric oxide has inhibitory effects on a variety of viral infections and its inhalation has been shown to be safe. University of British Columbia in collaboration with Mallinckrodt is conducting a study using inhaled gaseous nitric oxide antimicrobial treatment of COVID-19 infections (NCT03331445). Sanotize Research and Development Corp. in collaboration with the Emmes Company, LLC are planning to conduct a multicenter, randomized, controlled study to determine the efficacy of nitric oxide releasing solution treatment on the prevention and treatment of COVID-19 in healthcare workers and individuals at risk of infection (NCT04337918). PUL-042 is an inhalation solution consisting of a combination of two toll-like receptor ligands: Pam2CSK4 acetate, an agonist of TLR2 and TLR6, and a TLR9 agonist oligodeoxynucleotide with potential immunostimulating activity. Pulmotect, Inc. is conducting two clinical studies to evaluate the efficacy and safety of PUL-042 Inhalation Solution in reducing the severity of COVID-19 (NCT04312997; NCT04313023).

Convalescent Plasma

Convalescent plasma from patients who have recovered has been suggested to be safe and effective in SARS-CoV-2-infected patients. In an uncontrolled case series of 5 critically ill patients with COVID-19 and ARDS, convalescent plasma having neutralizing antibody showed an improvement in clinical status (15). A study conducted in two patients of COVID-19 with severe pneumonia and ARDS treated with convalescent plasma infusion showed favorable outcome (16). US Food and Drug Administration (FDA) announced on Mar. 24, 2020 that it is facilitating access to convalescent plasma, antibody-rich blood products that are taken from blood donated by people who have recovered from the COVID-19 virus, could shorten the length, or lessen the severity, of illness in COVID-19 patients. The classification of convalescent plasma as an investigational new drug by the FDA permits conducting clinical trials and compassionate use to treat patients with serious or life-threatening COVID-19 infections (17) via an emergency investigational new drug application (eIND). Red Cross has been asked by FDA to help identify prospective donors and manage the distribution of these products to hospitals that are treating COVID-19 patients.

Stem Cells

Experimental studies have demonstrated that mesenchymal stem cells (MSCs) or their exosomes (MSCs-Exo) significantly reduced lung inflammation and pathological impairment resulting from different types of lung injury. A pilot clinical study is being conducted using aerosol inhalation of the exosomes derived from allogenic adipose mesenchymal stem cells in the treatment of severe patients with novel coronavirus pneumonia (NCT04276987). Tianhe Stem Cell Biotechnologies Inc. has developed Stem Cell Educator (SCE) technology to reverse autoimmune response using human multipotent cord blood stem cells. SCE therapy is an attempt to restore immune balance and correct the overreaction of immune responses, the investigators therefore plan to treat COVID-19 patients with SCE therapy (NCT04299152). CAR-T (Shanghai) Biotechnology Co., Ltd. is conducting a clinical study to treat novel coronavirus induced severe pneumonia by dental pulp mesenchymal stem cells via an open, single center, single arm in 24 subjects (NCT04302519). A mesenchymal stem cell therapy produced by Cellavita is to assess its efficacy as an add-on therapy to standard treatment to treat patients with severe COVID-19 pneumonia (NCT04315987).

Inflammatory Response

COVID-19 patients with certain risk factors seem to die by an overwhelming reaction of the immune system to the virus, causing a cytokine storm with features of cytokine-release syndrome (CRS) and macrophage activation syndrome (MAS) and ARDS. There is evidence that cytokine-targeted therapies can improve outcomes in CRS or MAS. Neutralization of the inflammatory pathway induced by IL-6 may reduce mortality in patients with severe COVID-19 prone to CRS and ARDS. Tocilizumab (developed by Genentech, Roche), an anti-IL-6R biological therapy, has been approved for the treatment of CRS and is used in patients with MAS. It is hypothesized that it can reduce mortality in patients with severe COVID-19 prone to CRS and ARDS. The overall purpose of this study is to evaluate whether treatment with tocilizumab reduces the severity and mortality in patients with COVID-19. A multicenter, double-blind, randomized controlled phase II trial to determine the efficacy and safety of tocilizumab in the treatment of COVID-19 is being conducted in 100 patients (NCT04335071). Another randomized, double-blind, placebo-controlled, multicenter study to evaluate the safety and efficacy of tocilizumab in patients with severe COVID-19 pneumonia is in progress (NCT04320615).

Sarilumab, is being jointly developed by Regeneron and Sanofi, it is a fully human, monoclonal antibody that inhibits the IL-6 pathway by binding and blocking the IL-6 receptors. IL-6 may play a key role in driving the inflammatory response that leads to morbidity and mortality and patients with COVID-19 who develop ARDS. An adaptive phase II/III, randomized, double-blind, placebo-controlled study assessing efficacy and safety of sarilumab for hospitalized patients with COVID-19 is in progress of enrolling 400 patients (NCT04315298). Another study is in progress with the primary objective of evaluating the efficacy of sarilumab relative to the control arm in adult patients hospitalized with severe COVID-19 (NCT04327388). AstraZeneca would start a new clinical trial of acalabrutinib aimed at assessing it as a treatment for COVID-19. Acalabrutinib belongs to a class of drugs called Bruton's tyrosine kinase (BTK) inhibitors which can suppress autoimmune diseases and the trial will be to determine if it can prevent over reaction of the immune system producing cytokine storm in patients with COVID-19.

Piclidenoson is an anti-inflammatory agent that induces a robust anti-inflammatory effect, hence a trial has been proposed where hospitalized patients with COVID-19 will be randomized 1:1 to receive piclidenoson with standard care (intervention arm) or standard care alone (control arm) (NCT04333472). Tradipitant is an NK-1R antagonist being developed by Vanda Pharmaceuticals. A randomized, double-blind placebo-controlled trial to investigate the efficacy and safety of tradipitant to treat inflammatory lung injury associated with severe or critical COVID-19 infection is being planned (NCT04326426). OncoImmune, Inc. is conducting a phase III trial to determine efficacy of CD24Fc as a non-antiviral immunomodulator in COVID-19 treatment (NCT04317040). This trial will involve 230 patients randomized into blinded placebo and CD24Fc arms, with time to clinical improvement from severe to mild symptom as the primary endpoint. Swedish Orphan Biovitrum is sponsoring a phase II/III, randomized, open-label, parallel group, 3-arm, multicenter study investigating the efficacy and safety of intravenous administrations of emapalumab, an anti-interferon gamma (Anti-IFNγ) monoclonal antibody, and anakinra, an interleukin-1 (IL-1) receptor antagonist, versus standard of care, in reducing hyper-inflammation and respiratory distress in patients with SARS-CoV-2 infection (NCT04324021). I-Mab Biopharma Co. Ltd. is conducting a randomized, double-blind, placebo-controlled, multi-center trial to evaluate the safety and efficacy of TJ003234 (anti-GM-CSF monoclonal antibody) administered as an intravenous infusion in subjects with severe COVID-19 under supportive care, and to assess its effect on cytokines levels (NCT04341116). The potential for anti-viral activity by calpain inhibition in animal bleomycin lung injury models demonstrated that BLD-2660 (Blade Therapeutics) normalized tissue IL-6 levels. Therefore, a study to evaluate BLD-2660 as an add-on therapy to standard of care in hospitalized subjects with COVID-19 has been planned in 120 patients (NCT04334460).

The U.S. Food and Drug Administration on Apr. 10, 2020 issued an emergency use authorization for a blood purification system to treat patients with COVID-19 admitted to the ICU with confirmed or imminent respiratory failure. Spectra Optia Apheresis System and Depuro D2000 Adsorption Cartridge developed by Terumo BCT Inc. and Marker Therapeutics AG work by reducing the amount of cytokines and other inflammatory mediators associated with cytokine storm in the bloodstream by filtering the blood and returning the filtered blood to the patient.

Other Therapies

Corticosteroids have been tried in different scenarios of ARDS, including viral pneumonia, and the early use of dexamethasone appears to reduce the duration of mechanical ventilation in ARDS patients. A study to evaluate the effectiveness of dexamethasone compared to control (no corticosteroids) in ventilator-free days in patients with moderate and severe ARDS due to COVID-19 (NCT04327401).

A clinical study has been initiated by Tasly Pharmaceuticals, Inc. This is an open-label, randomized, blank-controlled treatment clinical study with an objective to investigate the effect of T89 (dantonic) on improving oxygen saturation and clinical symptoms in patients with non-critical type of COVID-19 pneumonia. The primary efficacy parameters include the time to oxygen saturation recovery to normal level (≥97%), the proportion of patients with normal level of oxygen saturation after treatment, and the total duration of oxygen inhalation (NCT04285190).

A study that will evaluate the efficacy and safety of Hydrogen-Oxygen Generator with Nebulizer (model AMS-H-03) developed by Shanghai Asclepius Meditech Co., Ltd. as an adjuvant therapy for the patients with COVID-19 infected pneumonia is recruiting patients (NCT04336462). This is an attempt to determine whether this device improves the clinical symptoms and reduces the incidence of severe pneumonia, as compared with the reference device of EverFlo Oxygen Concentrator (registration certificate No.: NMPA Registration Standard: 20162542389) manufactured by Respironics, Inc. USA.

Plasminogen has been reported to significantly increase in patients with ARDS and is important in degrading core components of the extracellular matrix including fibrin (18, 19). Intravenous plasminogen supplementation was effective in reducing premature infant ARDS and death (20-22). Since lungs from patients with COVID-19 have shown typical signs of ARDS, and hyaline membrane formation is mainly composed of fibrin, a study was conducted in 13 patients to determine whether plasminogen supplementation may be effective in treating lung lesions and hypoxemia during COVID-19 infections. Inhalation of plasminogen (10 mg dissolved in 2 ml sterile water) was given twice daily for severe and once daily for moderate COVID-19 patients. It was found that 5 patients showed improvement in density of ‘ground glass’ opacity and 6 patients showed improved oxygen saturation. This study has major limitation of lack of proper control group, however, it indicates a possible hope of combating critically ill patients with COVID-19 (23).

The coagulation and fibrinolytic systems have been considered to improve ARDS (24, 25). Plasminogen activators have been indicated in preclinical studies to attenuate ARDS progression and death (26, 27). It has been proposed that administration of tPA, as a compassionate approach, deserves merit because of high mortality in patients with COVID-19 suffering from ARDS (28). In a report of 3 cases where off-label intravenous administration of tPA (Alteplase) was carried out in patients with COVID-19 suffering from ARDS and respiratory failure. An improvement in PaO2/FiO2 (P/F) or SpO2/FiO2 (S/F) ratio was observed in all 3 patients ranging from 38% to 100%. However, improvements were transient and lost in all 3 patients after completion of tPA infusion (29). Moreover, a high risk of catastrophic bleeding from tPA must be carefully considered despite high mortality in COVID-19 patients with ARDS.

There exists a need to develop a method and pharmaceutical composition comprising centhaquine in a predefined amount and its analogues, and/or antiviral drugs, and/or supportive therapies to reduce fever, and/or anticoagulants for reduction of edema in the lungs, improvement in ratio of arterial partial pressure of oxygen to fraction of inspired oxygen (PaO2/FiO2 or SpO2/FiO2), blood oxygen saturation (SpO2), normalization in respiratory rate, reduction in lung infiltration, improvement in ARDS score, MODS and better blood flow and oxygenation of tissues to treat ARDS, multiple end organ failure and shock symptoms caused by coronaviruses infection, in particular SARS-CoV-2, MERS-CoV and SARS-CoV.

### SUMMARY

The present disclosure is envisioned towards a method and pharmaceutical composition comprising of centhaquine with or without antiviral therapies, convalescent plasma, stem cells or their exosomes, immunomodulation and cytokine-targeted therapies, blood purification systems, oxygen concentrator and generator, plasminogen supplementation, plasminogen activators, anticoagulants, steroids for improvement in ratio of arterial partial pressure of oxygen to fraction of inspired oxygen (PaO2/FiO2 or SpO2/FiO2), blood oxygen saturation (SpO2), normalization in respiratory rate, reduction in lung infiltration, improvement in ARDS score, MODS and better blood flow and oxygenation of tissues to prevent or treat ARDS, multiple end organ failure and shock symptoms caused by coronaviruses infection, in particular SARS-CoV-2, MERS-CoV and SARS-CoV.

### BRIEF DESCRIPTION OF DRAWINGS

These and other features, aspects, and advantages of the present disclosure will become better understood with regard to the following description, appended claims, and accompanying drawings where:

FIG. 1 illustrates a proposal to use centhaquine as an add-on treatment to provide hemodynamic stability, improve acute respiratory distress syndrome (ARDS), multiple organ dysfunction score (MODS) and reduce mortality.

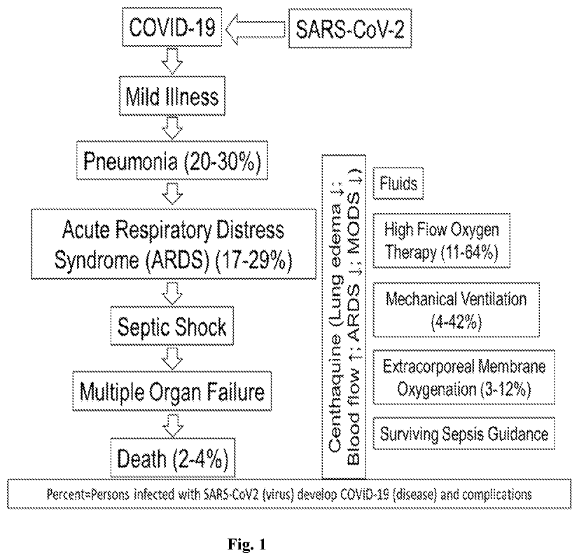

FIG. 2 illustrates a graphical representation of significant improvement in oxygen saturation (SpO2) of COVID-19 patients by intravenous administration of centhaquine in the dose of 0.01 mg/kg was observed;

FIG. 3 illustrates a graphical representation of Centhaquine improved SpO2/FiO2 in all 10 patients irrespective of age of the patient. Basal SpO2/FiO2 was found to be slightly poor in aged patients and the slope was −1.062, however, treatment with centhaquine started flattening the slope to −0.5905 at 2 hours and −0.2718 at 4 hours of treatment with centhaquine;

FIG. 4 illustrates a graphical representation of Centhaquine improved SpO2/FiO2 in COVID-19 patients. SpO2/FiO2 was found to improve following administration of centhaquine by 34.48 units within 2 hours and by 41.42 units in 4 hours;

FIG. 5 illustrates a graphical representation of Centhaquine improved clinical outcome of COVID-19 patients as determined by the WHO Ordinal Scale. Improvement started in 24 hours after treatment with centhaquine and at 72 hours of treatment a statistically significant (p=0.0169) improved was observed.

### DETAILED DESCRIPTION OF THE DISCLOSED EMBODIMENTS

As used herein, the term “an amount sufficient to” refers to amount that enables the achievement of the intended effect. Such an amount may be determined through various assays known in the art based on the intended effect. As used herein, the terms “applying” or “administering” refer to all means of introducing the specified agent, composition, or force to the specified region or subject. “Administration” or “application” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, inhalation, injection, and topical application. Administration can be for use in industrial as well as therapeutic applications. As used herein, the term “biodegradable” is used herein to describe substances, such as polymers, compositions, and formulations, intended to degrade during use. Biodegradable substances may also be “biocompatible,” i.e. not harmful to living tissue.

As used herein, the term “therapeutically effective amount” refers to a quantity sufficient to achieve a desired effect. In the context of therapeutic applications, the effective amount will depend on the type and severity of the condition at issue and the characteristics of the individual subject, such as general health, age, sex, body weight, and tolerance to pharmaceutical compositions. The skilled artisan will be able to determine appropriate amounts depending on these and other factors. In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment. The dose range of centhaquine could be from 0.00001 to about 1 mg/kg and may be administered once or multiple times in a day or in weeks or in months.

As used herein, the term “treating” or “treatment” includes preventing a disease, disorder or condition from occurring in a subject predisposed to or having a disease, disorder and/or condition; inhibiting the disease, disorder or condition, e.g., impeding its progress; and relieving or reversing the disease, disorder, or condition, e.g., causing regression of the disease, disorder and/or condition. Treating a disease or condition may also include ameliorating at least one symptom of the particular disease or condition.

The term “ARDS” refers to Acute respiratory distress syndrome (ARDS) is a type of respiratory failure characterized by rapid onset of widespread inflammation in the lungs (30). The signs and symptoms of ARDS often begin within two hours of an inciting event but can occur after 1-3 days. Signs and symptoms may include shortness of breath, fast breathing, and a low oxygen level in the blood due to abnormal ventilation (31). Other common symptoms include muscle fatigue and general weakness, low blood pressure, a dry, hacking cough, and fever (31).

In some embodiments, the basic composition, may be combined with remdesivir or lopinavir or ritonavir or arbidol or favipiravir or ribavirin or interferon beta-1B or alpha-interferon or mesenchymal stem cells or their exosomes or chloroquine or chloroquine phosphate or hydroxychloroquine or pirfenidone or antibodies like REGN3048 and REGN3051 or mRNA-1273 or bevacizumab or bromhexine or fingolimod or T89 or eculizumab or carrimycin or oxygen treatment or corticosteroids or methylprednisolone or inhaled nitric oxide gas or losartan or darunavir or tocilizumab or tetrandrine or aviptadil or thalidomide or sarilumab or vitamin C or plasma therapy.

Preclinical and clinical studies have demonstrated that centhaquine effectively addresses the major challenges associated with COVID-19. First, studies in swine model of shock centhaquine significantly reduced pulmonary edema and improved The Horowitz index the PaO2/FiO2 ratio. Second, improved tissue blood perfusion by centhaquine can rapidly clear inflammatory cytokines and prevent oxidative and apoptotic damage. Third, in phase III clinical trial centhaquine was effective in reducing ARDS and MODS. Fourth, in clinical studies centhaquine statistically significantly reduce mortality of patients.

Centhaquine) is a first-in-class resuscitative agent that is final stages of approval in India. Centhaquine acts through a unique mechanism of action that is completely different from any of the existing resuscitative agents. It increases blood pressure and cardiac output by augmenting venous blood return to the heart (venous alpha2B-adrenergic receptor stimulation) (32-36). It also produces arterial dilation by acting on central α2A-adrenergic receptors to reduce sympathetic activity and systemic vascular resistance (37). A significant number of patients with COVID-19 are admitted to the ICU and many of them are intubated and kept on positive pressure ventilation. A very high mortality is associated with patients who are on ventilator support. About 30% of patients encounter life-threatening hypotension due a decrease in venous return to the heart following endotracheal intubation and/or positive pressure ventilation (38, 39). As a result of its unique mechanism of action, centhaquine is expected to attenuate positive pressure ventilation induced decrease in venous return to the heart and prevent life-threatening hypotension. Centhaquine is likely to provide hemodynamic stability, improve tissue oxygenation, reduce pulmonary edema, reduce ARDS, reduce MODS and decrease mortality in COVID-19 patients.

Recently, guidelines on the management of critically ill adults with COVID-19 were published (40, 41). These guidelines authored by 36 experts from 12 countries were developed by The Surviving Sepsis Campaign (SSC). They have been grouped in four categories: (1) infection control and testing; (2) hemodynamic support; (3) ventilatory support and (4) therapy. It is recommended that acute resuscitation of adults with shock be done with a conservative fluid administration and preferring crystalloids over colloids. Norepinephrine has been suggested as the first-line vasoactive, adding vasopressin as a second-line agent is suggested if the target (60-65 mmHg) mean arterial pressure cannot be achieved by norepinephrine alone (40, 41). Acute hypoxemic respiratory failure despite conventional oxygen therapy requires close monitoring, and if worsening occurs an early intubation along with positive pressure ventilation is recommended. A mortality rate in the intensive care unit (ICU) of COVID-19 patients has been reported to be more than 79% (42). It has been found that using centhaquine as a resuscitative agent in shock (hypovolemic) significantly reduced 28-day all-cause mortality from 11.76% in patients receiving standard treatment to 2.94% in patients that received centhaquine (P=0.0742). In a metanalysis of phase II and III trials of centhaquine in hypovolemic shock mortality was reduced from 10.71% to 2.20% (Odds ratio 5.340, 95% CI 1.27-26.50, p=0.0271). It is quite likely that centhaquine as a resuscitative agent will help patients with COVID-19 and reduce mortality.

The outbreak of COVID-19 disease which is evolving and expanding at a rapid pace has created major challenges to resuscitation efforts. Critically ill COVID-19 patients are managed for ARDS and continued intensive care management. Patients with COVID-19 usually have hypovolemia and fluids are administered with caution keeping in mind pre-load responsiveness. A high incidence of myocardial dysfunction has been reported in COVID-19 patients (43-45). Using centhaquine as a resuscitative agent can be beneficial because it has also been shown to be highly effective in a swine model of in hospital cardiac arrest (46).

An improved blood perfusion will enhance the clearance of toxic cytokines produced as a result of overactive immune reaction in patients with COVID-19. Plasma cytokine levels depend on several factors: the intensity of production, the number of cell receptors availability, the clearance of cytokines, the affinity of the receptors for cytokines (47). Centhaquine can help and promote rapid clearance of these cytokines. It will be particularly useful when centhaquine is combined with various agents that are either available or being developed to counter the overwhelming reaction of the immune system to the virus, causing a cytokine storm. Blood purification systems to remove cytokines such as high-volume continuous hemofiltration or cytokine and/or endotoxin removal have been suggested but with little success (47). There are several methods being developed to remove cytokines from blood circulation using devices such as Cytosorb (extracorporeal cytokine removal), Hemofeel (continuous venovenous hemodiafiltration) and EMiC2 (continuous venovenous hemodialysis) (48, 49). Most of these devices are extremely expensive, complicated to operate, and are available only at a limited number of institutions. Centhaquine increases stroke volume, cardiac output (32, 36, 50, 51) and blood flow to the vital organs, prevents organ failure and improves survival in rat, rabbit and swine models of hypovolemic shock (32, 36, 50, 51). Enhancing tissue perfusion is a significant advantage in reducing the volume of resuscitation and preventing extravasation of fluid and adverse effect of lung edema. Centhaquine does not act on beta-adrenergic receptors, and therefore the risk of arrhythmias is alleviated. Centhaquine has several advantages because improved tissue blood perfusion will not only remove toxic cytokines but also provide oxygenation and nutrition to the tissues. Since there are limited therapeutic options for this life-threatening condition, centhaquine may fulfil the unmet need for serious, life-threatening condition of COVID-19 during this pandemic outbreak. Centhaquine is likely to restore the immune balance and correct the overreaction of immune responses in patients with COVID-19 that develop cytokine storm.

Studies in a swine model of shock showed that, centhaquine significantly reduced pulmonary edema and improved Horowitz index (ratio of partial pressure of oxygen in blood and the fraction of oxygen in the inhaled air (36).

Improvement in ARDS and MODS: In randomized, controlled, multicentric clinical trial patients (N=155) with hypovolemic shock, centhaquine significantly improved ARDS scores and MODS score (MODS). In a phase 3 study of hypovolemic shock, ARDS and MODS were secondary endpoints and they were both achieved with a significant p-value with centhaquine (33, 52).

ARDS in Shock Patients (N=105): Acute Respiratory Distress Syndrome (ARDS) was compared between day 1 (before resuscitation) and day 3 of resuscitation. In control patients receiving standard treatment the difference between means was 0.04839±0.05696 (P=0.4023). On the other hand, in centhaquine treated group the ARDS difference between means was 0.1065±0.04464 (P=0.0202). These results indicate that centhaquine treatment significantly improved ARDS following resuscitation, whereas in control group there was insignificant improvement.

MODS in Shock Patients (N=105): Multiple Organ Dysfunction Score (MODS) was compared between day 3 and day 7 of resuscitation. There was no improvement in MODS in the control group and the difference between means was 0.00±0.2697 (P>0.999), whereas in centhaquine group the difference between means was 0.9091±0.1964 (P=0.0001). Centhaquine treatment significantly decreased MODS whereas in control the improvement was not significant.

Centhaquine has been evaluated for its safety, sensitivity and toxicity in various species for single and multiple doses and acute as well as chronic exposure (33). Centhaquine has been found to be safe and well tolerated in preclinical and clinical studies. Its safety has also been demonstrated in a Phase I study (NCT02408731) in 25 human subjects (53, 54). There were NO adverse events related to centhaquine reported in phase II (NCT04056065) and phase III (NCT04045327) clinical studies.

Results of clinical phase II study (CTRI/2017/03/008184; NCT04056065) indicate that, centhaquine is a novel, first-in-class, highly effective resuscitative agent for hypovolemic shock as it demonstrated highly significant efficacy in improving blood pressure (p<0.0001), lactate levels (p=0.0012), base-deficit (p<0.0001), reduction in use of vasopressors and reduced mortality (33, 55-57). In a 105-patient randomized, blinded, multicenter study (CTRI/2019/01/017196; NCT04045327) a total of 34 (22 male and 12 female) patients in control and 68 (41 male and 27 female) patients in centhaquine groups completed the study. Blood lactate levels at day 3 of resuscitation were found to be significantly lower in centhaquine group compared to control group receiving standard treatment (P=0.046). Base-deficit improved in patients treated with centhaquine by 1.430±1.047 mmol/L compared to control patients receiving standard treatment (33). In total 180 human subjects have been studied (combined phase I, II and III), out of which 155 were patients with hypovolemic shock. Centhaquine reduced the mortality from 9.68% in patients receiving standard treatment to 2.15% in patients that received centhaquine (odds ratio 4.875; 95% CI 1.162-24.18; P=0.0190).

Results of phase II and phase III clinical studies indicate that, centhaquine treatment can provide hemodynamic stability and prove to be beneficial in improving ARDS, MODS and shock symptoms in patients infected with COVID-19. Centhaquine can reduce morbidity and mortality in COVID-19 by reduction of edema in the lungs, improved ARDS scores and better oxygenation of tissues.

Centhaquine has been evaluated for its safety, sensitivity and toxicity in various species for single and multiple doses and acute as well as chronic exposure (33). Centhaquine was found to be safe and well tolerated in healthy human subjects (53, 54). Safety and efficacy of centhaquine is established (Phase I, phase II and phase III clinical studies)

Centhaquine has shown efficacy in improving ARDS, MODS and survival in serious and life-threatening condition of hypovolemic shock and it has the potential to improve morbidity and mortality in patients with COVID-19. Preclinical and clinical studies have demonstrated that centhaquine effectively addresses the major challenges associated with COVID-19.

First, studies in swine model of shock centhaquine significantly reduced pulmonary edema and improved The Horowitz index the PaO2/FiO2 ratio. Second, improved tissue blood perfusion by centhaquine can rapidly clear inflammatory cytokines and prevent oxidative and apoptotic damage. Third, in phase III clinical trial centhaquine was effective in reducing ARDS and MODS. Fourth, in clinical studies centhaquine statistically significantly reduce mortality of patients.

Centhaquine has shown efficacy in improving ARDS, MODS, and survival in a serious life-threatening condition of hypovolemic shock; hence, it can improve morbidity and mortality in patients with COVID-19. Preclinical and clinical studies have demonstrated that centhaquine effectively addresses the major challenges associated with COVID-19. Studies in the swine model of shock, centhaquine significantly reduced pulmonary edema and improved The Horowitz index (PaO2/FiO2 ratio). Improved tissue blood perfusion by centhaquine can rapidly clear inflammatory cytokines and prevent oxidative and apoptotic damage. In phase III clinical trial, centhaquine was effective in reducing ARDS and MODS. In clinical studies, centhaquine statistically significantly reduce the mortality of patients. We propose using centhaquine at a dose of 0.01 mg/kg, along with the standard of care, to be administered to patients meeting the eligibility criteria. There will be no change in the current standard of care of critically ill COVID-19 patients. Patients will continue receiving standard of care, and centhaquine will be an add-on treatment to provide hemodynamic stability and improve ARDS, MODS scores and reduce mortality.

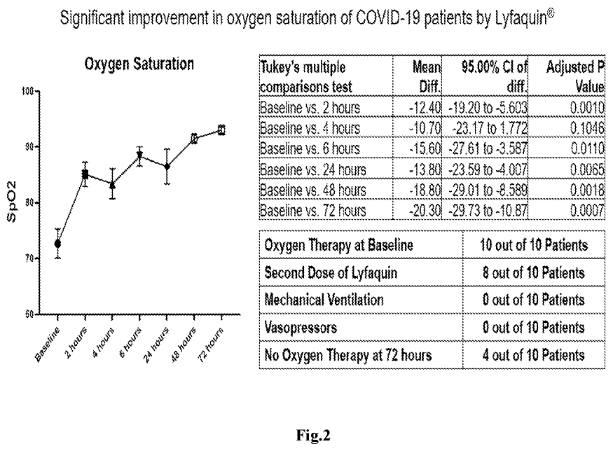

The effect of centhaquine (Lyfaquin®) was determined COVID-19 patients. A significant improvement in oxygen saturation (SpO2) of COVID-19 patients by intravenous administration of centhaquine in the dose of 0.01 mg/kg was observed (FIG. 2). An improvement of SpO2 improved by 12.40 units within 2 hours of administration of centhaquine and further treatment with centhaquine led to an improvement of SpO2 by about 20 units. Four out of 10 patients did not even need oxygen therapy at 72 hours of treatment with centhaquine.

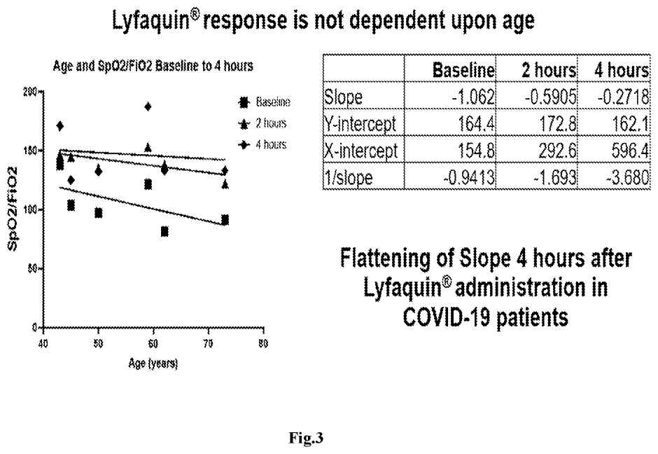

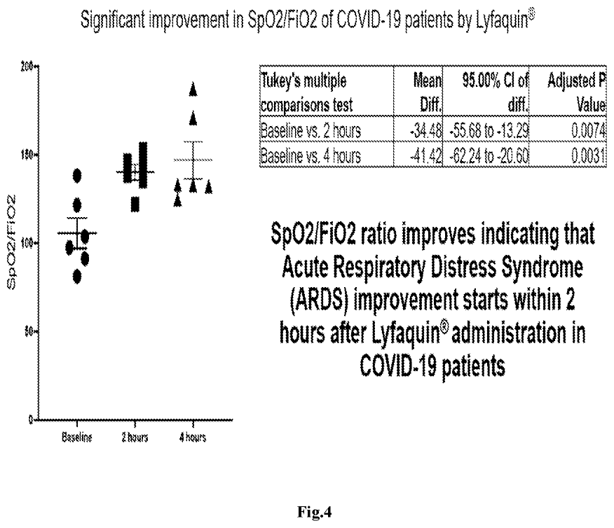

We also determined the effect of age on the improvement in ratio of SpO2 and FiO2 (SpO2/FiO2) after administration of centhaquine (FIG. 3). We found that centhaquine improved SpO2/FiO2 in all patients irrespective of age of the patient. Basal SpO2/FiO2 was found to be slightly poor in aged patients and the slope was −1.062, however, treatment with centhaquine started flattening the slope to −0.5905 at 2 hours and −0.2718 at 4 hours of treatment with centhaquine (FIG. 3). Centhaquine (Lyfaquin®) improved SpO2/FiO2 in COVID-19 patients. SpO2/FiO2 was found to improve following administration of centhaquine by 34.48 units within 2 hours and by 41.42 units in 4 hours (FIG. 4).

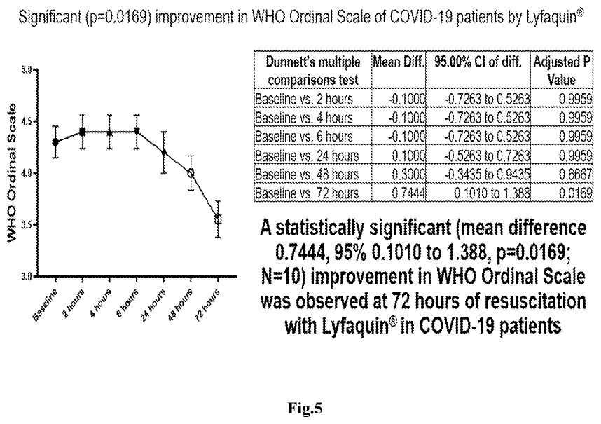

WHO Ordinal Scale was used to determine whether centhaquine (Lyfaquin®) improved the outcome of patients with COVID-19 (FIG. 5). A statistically significant (mean difference 0.7444, 95% 0.1010 to 1.388, p=0.0169; N=10) improvement in WHO Ordinal Scale was observed at 72 hours of resuscitation with centhaquin (Lyfaquin®) in COVID-19 patients.

While the disclosed embodiments have been described with reference to illustrative embodiments, this description is not intended to be construed in a limiting sense. Various modifications and combinations of the illustrative embodiments as well as other embodiments of the present disclosure, will be apparent to persons skilled in the art upon reference to the description. It is, therefore, intended that the appended claims encompass any such modifications or embodiment.

What is claimed is:

1. A pharmaceutical composition for treating acute respiratory distress syndrome, comprising:
(a) centhaquine or its analogues in a predefined amount;
(b) antiviral therapies for SARS-CoV-2 infection (remdesivir, ivermectin, chloroquine, hydroxychloroquine, azythromicyn, tenofovir, emtricitabine, ritonavir, lopinavir, ASC09, favipiravir, danoprevir, angiotensin-converting enzyme inhibitors (ACEI), angiotensin receptor blockers (ARB), recombinant human angotensin-converting enzyme 2 (rhACE2), xiyanping, alpha-interferon, fludase (DAS181), eicosapentaenoic acid free fatty acid (EPA-FFA), nitric oxide, PUL-042, Pam2CSK4 acetate, agonists of TLR2 TLR6, and TLR9), convalescent plasma, stem cells or their exosomes, immunomodulation and cytokine-targeted therapies (itolizumab, tocilizumab, sarilumab, acalabrutinib, piclidenoson, tradipitant, CD24Fc, emapalumab, anakinra, TJ003234, BLD-2660, blood purification systems, Spectra Optia Apheresis System, corticosteroids) oxygen concentrator and generator, T89, dantonic, plasminogen supplementation, plasminogen activators, and alteplase;
(c) budesonide, supportive therapies to reduce fever (like acetaminophen or ibuprofen), steroids (dexamethasone, prednisolone);
(d) anticoagulants (aspirin, heparin, non-heparin anticoagulants such as argatroban, bivalirudin, danaparoid, fondaparinux or a direct oral anti-coagulant (DOAC);
(e) inhaled synthetic surfactant, antibody to endotoxin, interferon-beta-1a, IV prostaglandin E1, neutrophil elastase inhibitors, nitric oxide; and
(f) an excipient.

2. A method for reduction of edema in the lungs, improvement in ratio of arterial partial pressure of oxygen to fraction of inspired oxygen (PaO2/FiO2 or SpO2/FiO2), blood oxygen saturation (SpO2), normalization in respiratory rate, reduction in lung infiltration, improvement in ARDS score, MODS and better blood flow and oxygenation of tissues using centhaquine or its analogues.

3. The method of claim 2, wherein centhaquine and/or its analogues are delivered intravenously, orally, intramuscularly, subcutaneously by procedures through direct injections, osmotic mini-pumps and reciprocating perfusion systems.

4. The method of claim 2, wherein centhaquine or its analogues are conjugated with either microparticles or nanoparticles.

5. The method of claim 2, wherein centhaquine or its analogues dose range is about 0.00001 to 1 mg/kg.

6. The method of claim 2, wherein the dosage of centhaquine or its analogues may be administered once or multiple times in a day or in weeks or in months.
